(19) United States
(12) Patent Application Publication
(10) Pub. No.: US 2018/0112168 A1
(43) Pub. Date: Apr. 26, 2018

(54) Organ Production

(71) Applicant: Doyka Diaz, Lakeland, FL (US)

(72) Inventor: Doyka Diaz, Lakeland, FL (US)

(21) Appl. No.: 15/789,810

(22) Filed: Oct. 20, 2017

Related U.S. Application Data
(60) Provisional application No. 62/410,833, filed on Oct. 20, 2016.

(51) Int. Cl. C12M 3/00; C12N 5/071

(52) U.S. Cl. CPC C12M 21/08; C12N 5/0697

(57) ABSTRACT

All chambers, protein or separation columns, sensors, computer systems, smart technology, feedback mechanisms, ships, membranes, pumps, valves, recipient, scanning, and quantifiers or device used will be constructed with material approved by the FDA and not rejected by the organism. Materials or tools not approved by FDA could only be used for construction purposed not for insertion in the organism (eg. nanotechnology). With these conditions organs will be designed to be used in the body or outside de body. The Organs Production system could be used in humans, animals, or technology.

### BACKGROUND OF THE INVENTION

### Previews Art

First previous art: in Sep. 2, 2014 U.S. Pat. No. 8,822,535 talks about survivability of cells, tissues, organs and organisms. This is different than the Organ Production System because what it does is to preserve cells, tissues, organs and organisms. It is about survivability and not Production. This previous art is about manufacturing for enhancing survivability. The Organ Production System is patentable because it is about production of living like functions. The Organ Production System is not a product to enhance living cell unit's survivals for transplant. The Organ Production System is the creation of organs from approved materials.

Second previous art: in Mar. 21, 2017 U.S. Pat. No. 9597321 inventors Paniker; Bijoy, Mishra; Rama K., Lim; Dong Sung, Oehlen; Lambertus J. W. M. talks about Antifibrotic compounds and the use thereof. It talks about solution or treatments for abnormal or excessive fibrosis. The Organ Production System is different than this previous art because it is producing units without provoking abnormal fibrosis.

Third previous art: in Mar. 21, 2017 U.S. Pat. No. 9598477 inventor Yamashita; Hitoshi, Kusudo; Tatsuya talks about the use of adipocytes differentiation. The Organ Production System is different than this previous art because it is not talking about a transformation or differentiation. The Organ Production System makes the units from non-living material.

The Organ Production System is patentable because, there is not previous art that produces units with non- living materials capable of living functions.

Fourth previews art: Dec. 13, 2016 U.S. Pat. No. 9,517,295 Blood treatment systems and methods by

Inventors Wilt; Michael J. (Windham, N.H.), van der Merwe; Dirk A.(Canterbury, N.H.), Ballantyne; Todd A. (Amherst, N.H.), Grant; Kevin L. (Litchfield, N.H.), Rosse; Jonathan P. (Amherst, N.H.), Flynn; Catharine N. (Manchester, N.H.), Yee; Brian K. (Manchester, N.H.) is a dialysis that has monitors at least conductivity and temperature of dialysate in the flow circuit upstream of a dialyzer and enters a fail-safe state if the measured conductivity is outside of a range of values. And exposing sensors in fluid paths to temperatures or conductivities that are outside pre-determined permissible ranges of values, and confirming that the FPGA safety system enters a fail-safe state in response to the temperatures or conductivities. The FPGA may be configured to monitor an amount of ultrafiltration fluid withdrawn from a patient during dialysis treatment and to enter a fail-safe state if a maximum amount is exceeded.

Although, this system has sensors and monitors for alerts The Organ production system is still patentable for the innovation of monitors that will be controlled and connected to smart technology for the machine to solve the problem automatically in vivo inside the patient functioning as a regular organ all day long. The organ production system is not a dialysis system it is an organ replacement unit with sensors controls that are automated for feed-back mechanism controlled by a smart computer software, it has columns that filtrate proteins, osmosis and diffusion chambers pre-arranged for filtration to happen with nanotechnology designed membranes and using only in-vivo materials that are permitted by the FDA.

### DEFINITION

Organ production systems is a way of making artificial organs for survival of organisms. They are made from scratch using approved materials from the FDA that could be organic or not organic. The idea is using these organs to replace old organs in vivo or during surgical procedures. They could be used for robotics as well.

### SUMMARY

This is a system to make organs like the kidney with osmotic chambers, protein or separation columns, sensors, computer systems, smart technology, feedback mechanisms, ships, membranes, pumps, valves, recipient, scanning, and quantifiers or device used will be constructed with material approved by the FDA and not rejected by the organism. Materials or tools not approved by FDA could only be used for construction purposed not for insertion in the organism (eg. nanotechnology). With these conditions organs will be designed to be used in the body or outside de body. The Organs Production system could be used in humans, animals, or technology.

### DRAWINGS EXPLANATIONS

FIG. 1: Is the basic representation of a kidney unit

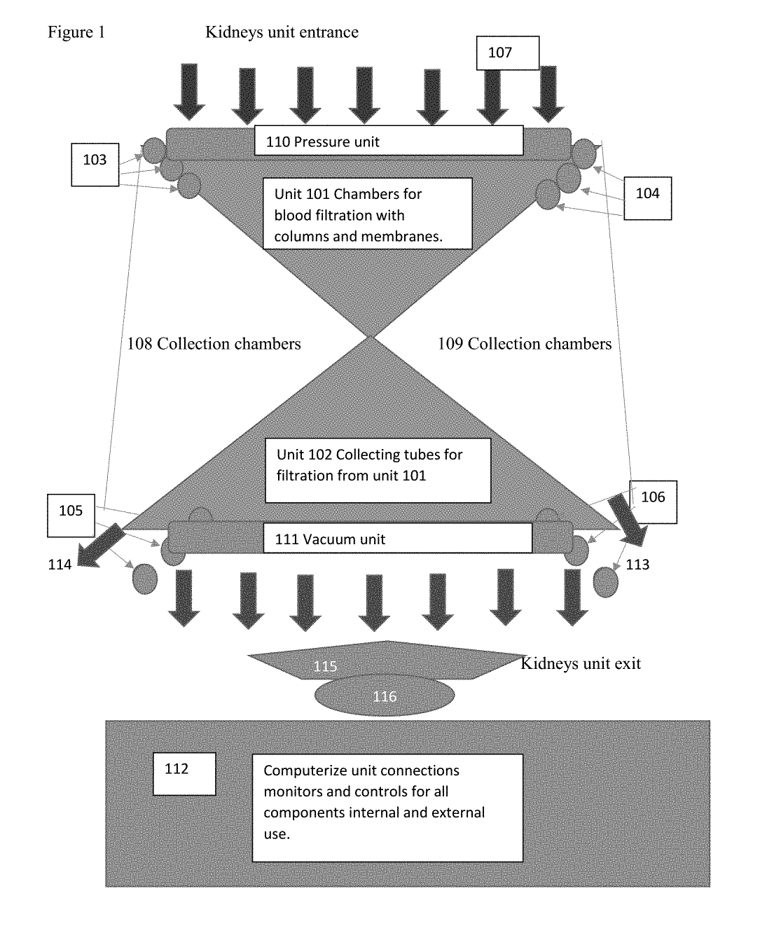

101 is a chamber for blood filtration with columns to separate the necessary proteins. This chamber will also have membranes with the right openings some produced with nanotechnology enabling the fluid of liquids to separations chambers according to osmotic pressure changes and sizes. Here is where you will find the osmosis and diffusion pre-set units with the appropriate concentrations for filtration. In this unit most blood filtration will happen.

102 will collect all the none reusable material from unit 101 to deposit in the urinary track.

108 and 109 will serve as reabsorption chambers for water, proteins and all reusable material that would come out of the 101 chambers after separation.

103,104,105 and 106 are monitor like units for collection of data to control the system using sensors and cameras. These units will report all data collected to 112.

112 will collect data processed it and react with a feedback mechanism actions to control the system parameters like: temperature, pressure, flow, salinity, concentration of proteins, water, conductivity, stoichiometry sensors. This system will be connected to each part of the kidney unit and control it via feedback mechanism using data from all kinds of sensors and cameras. Including the stoichiometry computerized sensors.

110 will create and release pressure in the system as needed according to the feedback and orders received from unit 112.

111 will create the vacuum needed for the correct and appropriate flow of the system for the right velocity and amount of filtration needed for the current or at the specific moment in vivo activity. Will react and function according to the feedback from 112 and will be coordinated with the rest of the system to maintain parameters at the right level.

107 is the entrance to the system

113 and 114 will be the connection back to the blood system to bring reusable materials with clean blood back into the body.

115 will be the collector that will bring the urine materials back to the ureters and urine bladder after blood filtration.

116 will be the sphincter like unit that will act for openings and closings of each valve or opening connecting all units.

### SPECIFICATIONS: DETAILED DESCRIPTION

1—Organ production systems is a way of making artificial organs for survival of organisms. They are made with a system of chambers where the steps for the realization of the organs functions are performed. This will be connected to recipients with the final product from each of the organs chambers steps. The steps and entries or released from each chamber recipient or organ can be controlled by valves. The valves and the regulation of the system could be also regulated by a computerized system for internal or external control when necessary.
2—Each chamber can have valves that control entry and pressure when necessary
3—Each valve can have an external controller in a computer to be adjusted if necessary by the surgent or Dr.
4—Each chamber or valve can have sensors that report and control the integrity of the system
5—Each sensor can be connected to the computerized system for reading and control of the organ unit.
6—The sensors, valves, and specific organ steps chambers would be monitored and reported to the internal as well as external computerized system for prevention of failure.
7—The system could include microphones and cameras for monitoring performance.
8—Depending on the king of organ the chambers would have different make up qualities
9—Chambers will have membranes, buffers, enzymes, columns or any other devices as needed to perform the function of the organ in a balanced closed or opened system.
10—The organs system will be capable of functioning alone as well as with internal or external regulations.

What is claim is:

1. A system to create any kind of organs using materials that are less likely to be rejected by the organisms using technologies.

2. Organs in claim one could use pre-existing organs or they can be designed from scratch.

3. Organs in claim 1 would be connected to the organism system to function.

4. Organs from claim one would be made using many technologies that might not be introduced in the organism.

5. Organs from claim one will perform the basic function of the regular organism organs to help the living system survive.

6. Organs from claim one could be use as artificial in surgery salon care as well as in vivo artificial organs for the well-being of the organism.
